(19) United States
(12) Patent Application Publication
(10) Pub. No.: US 2025/0082224 A1
(43) Pub. Date: Mar. 13, 2025

(54) A FILTER SUPPORT SYSTEM FOR HOMOGENEOUS WRINKLE DISTRIBUTION ON MOUTHPIECES THAT SHRINK WHEN COOLED DOWN DURING THE PRODUCTION PROCESS

(71) Applicant: INOFAB SAGLIK TEKNOLOJILERI ANONIM SIRKETI, Ankara (TR)

(72) Inventors: Merthan ÖZTÜRK, Çankaya/Ankara (TR); Kerem YASAR, Çankaya/Ankara (TR); Ahmet OGUZ, Çankaya/Ankara (TR); Kadir Tayyip TASLICA, Çankaya/Ankara (TR); Ilayda ALTUN, Çankaya/Ankara (TR); Selim YÖNET, Çankaya/Ankara (TR); Çagkan KARAARSLAN, Çankaya/Ankara (TR); Ceren YAVUZ, Çankaya/Ankara (TR)

(73) Assignee: INOFAB SAGLIK TEKNOLOJILERI ANONIM SIRKETI, Ankara (TR)

(21) Appl. No.: 18/728,349

(22) PCT Filed: Dec. 23, 2022
(86) PCT No.: PCT/TR2022/051591

§ 371 Date: Jul. 11, 2024

(30) Foreign Application Priority Data
Jan. 14, 2022 (TR) 2022/000476

(51) Int. Cl. A61B 5/097; A61B 5/00; A61B 5/087

(52) U.S. Cl. CPC A61B 5/097; A61B 5/087; A61B 5/682

(57) ABSTRACT

The present invention relates to a method for allowing the filters for protecting against contamination during the passage of the air flow by the sensors in pulmonary function tests to homogeneously distribute the wrinkles of the mouthpieces during production. The present invention relates to the solution of the problems experienced because the filter does not shrink or sufficiently shrink the aperture where the filter is located due to the shrinkage of the mouthpiece taken from the mold produced after melting the plastic raw material in the mold, which protects the transceivers from contamination.

### TECHNICAL FIELD

The present invention relates to a method for allowing the filters for protecting against contamination during the passage of the air flow by the sensors in pulmonary function tests to homogeneously distribute the wrinkles on the mouthpieces during production.

### BACKGROUND

One of the methods used during the pulmonary function tests performed with spirometers is the comparison of the flight times of the signals published and read by two reciprocally positioned transceivers, which are angled with an airway through which air flow takes place. Various spirometers operating according to this method are described in documents U.S. Pat. Nos. 5,419,326, 5,647,370, JP2013250254, US2010145213, and WO2019004966. Interchangeable mouthpieces extending along the airway are used to perform respiratory function tests between patients without contamination.

However, the closest patent application in the literature is a patent application numbered 2020/01739 and it is related to the method before this application. In the aforementioned application, the lens effect of wrinkling on the sound wave is mentioned.

During tests performed with spirometers or other pulmonary function test devices, there are windows on the wall of the airway corresponding to transceivers and acting permeably at ultrasonic frequencies so that the signals can interact with the air flow in a healthy way. During the tests, there are filters covering the windows to protect the transceivers from the risk of contamination. The filters can transmit ultrasonic signals, especially porous ones. The porous structure is preferably obtained by weaving micrometer-level yarns.

Woven filters can take random wrinkled forms if they are placed on a drawing material before pulling after being taken from the mold due to small changes in production conditions. These filters used on the mouthpieces of spirometers can change the air flow rate, shape, and cross-sectional area during respiratory function tests. As a result of these changes, the path of the signal in the mouthpiece may vary. When the shape/distribution of the filter wrinkle in each mouthpiece is uncontrolled, the mouthpieces produced in series can show different measurement results when they are subjected to the same air flow.

In order to solve this problem, EP3403813A1 describes the tensioning of the filter by thermal processes after the production of the mouthpiece. However, it is estimated that the stretching process will bring additional processing time, additional costs, and additional production complications. The ability to package products taken directly from the mold after control and sterilization without the need for additional operations will reduce and accelerate production costs.

### THE OBJECT AND BRIEF DESCRIPTION OF THE INVENTION

The present invention relates to the solution to the problems experienced because the filter itself does not shrink or sufficiently shrink the aperture where the filter is located due to the shrinkage of the mouthpiece taken from the mold produced after melting the plastic raw material in the mold, which protects the transceivers from contamination.

The object of the invention is to ensure that wrinkle or corrugated characteristics of all mouthpieces produced in succession during mass production perform low deviating wrinkles without showing great differences. Measurement standards indicate that the measurement values taken from different mouthpieces may contain a maximum of 3% difference from each other or may measure the flowing air volume with a maximum of 50 ml error. Thanks to the support of the filters of the mouthpiece of the invention, it is aimed to double or even triple the sensitivity of these values.

The filter, which is shrunk/crumpled by the shrinking mouthpiece and placed plain at the beginning, will be positioned in the relatively smaller new aperture area by forming two basic forms. These forms are observed as follows:
multiple uncontrolled scattered peaked wavy wrinkled structures within the aperture,
the curved structure formed by the root mean square of the top and bottom points of the waves.

The invention aims to control both the wavy wrinkled structure and the bump consisting of waves and to give repeatable results in mass production.

### DEFINITIONS OF FIGURES DESCRIBING THE INVENTION

The figures and related descriptions used to better explain the wrinkle-managed filter on the mouthpiece developed by this invention are as follows.

FIG. 1 A view of the filter mounted before shrinkage occurs during the initial installation of the filter without the support line during the production of the mouthpiece.

FIG. 2 An AA-Sectional view of the mounted version of the filter before shrinkage occurs during the initial installation of the filter without the support line during the production of the mouthpiece.

FIG. 3 A view of the filter mounted after shrinkage occurs during the first installation of the filter without the support line during the production of the mouthpiece.

FIG. 4 A BB-Sectional view of the mounted version of the filter after shrinkage occurs during the first installation of the filter without the support line during the production of the mouthpiece.

FIG. 5 A view of the filter mounted before shrinkage occurs during the first installation of the filter with the support line during the production of the mouthpiece.

FIG. 6 A CC-Sectional view of the mounted version of the filter before shrinkage occurs during the first installation of the filter with the support line during the production of the mouthpiece.

FIG. 7 A view of the mounted filter after shrinkage occurs during the first installation of the filter together with the support line during the production of the mouthpiece.

FIG. 8 A DD-Sectional view of the mounted version of the filter after the shrinkage occurs during the first installation of the filter with the support line during the production of the mouthpiece.

FIG. 9 A perspective view of the entire mouthpiece with filter supports for the preferred use of the support lines.

FIG. 10 A perspective view of the state where the filter is not supported on the mouthpiece.

FIG. 11 A GG-section of an alternative situation that shows that the filters become outwardly oriented while the molded and cooled mouthpiece shrinks since the filter is not supported on the mouthpiece.

FIG. 12 Another GG section of the alternative situation showing that the filters become inwardly oriented while the cooled mouthpiece is shrinking and taken from the mold since the filter is not supported on the mouthpiece.

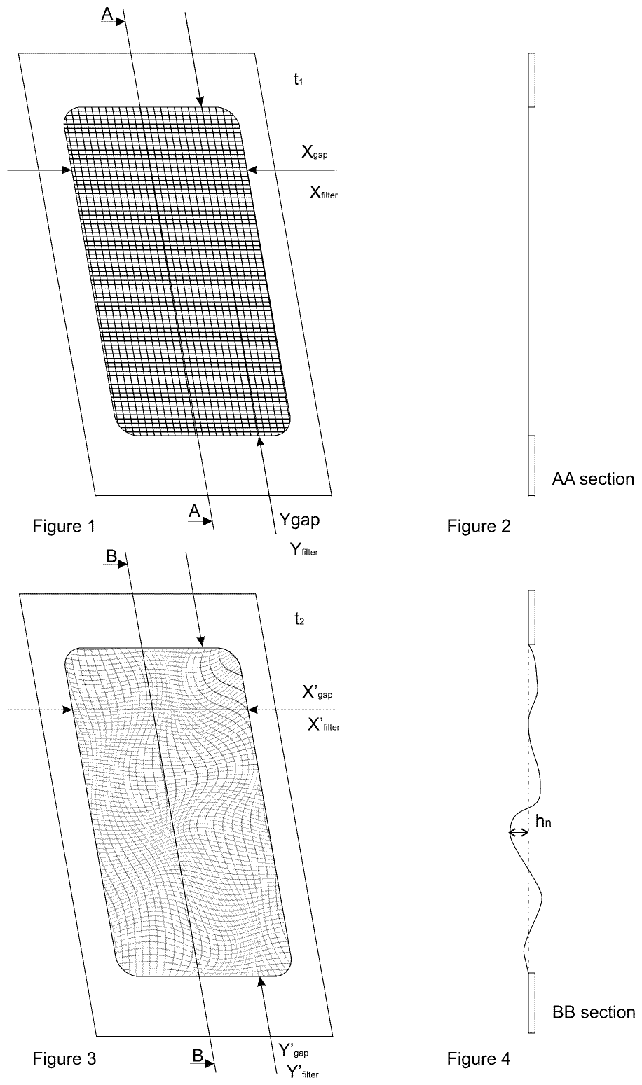

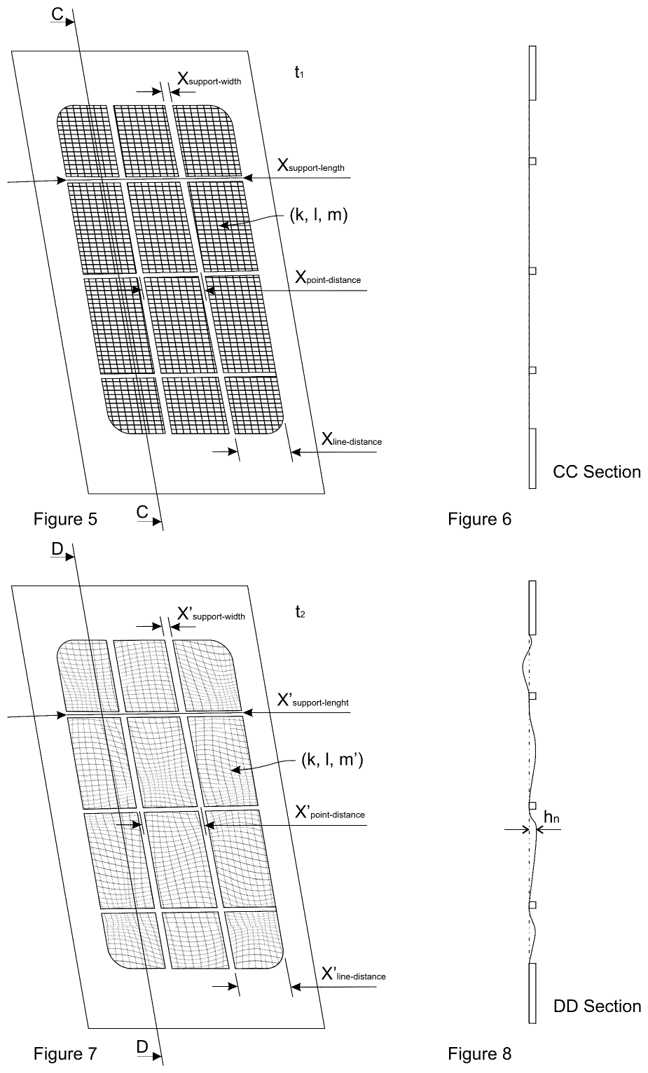

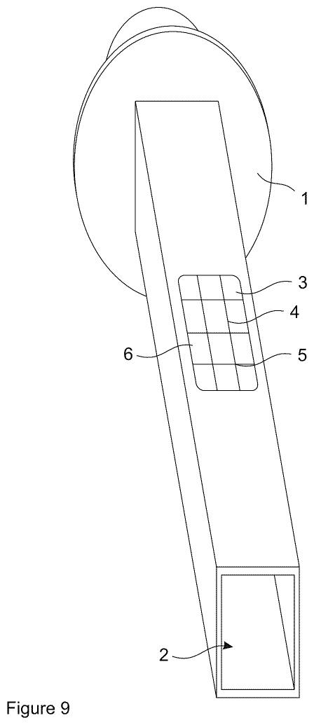

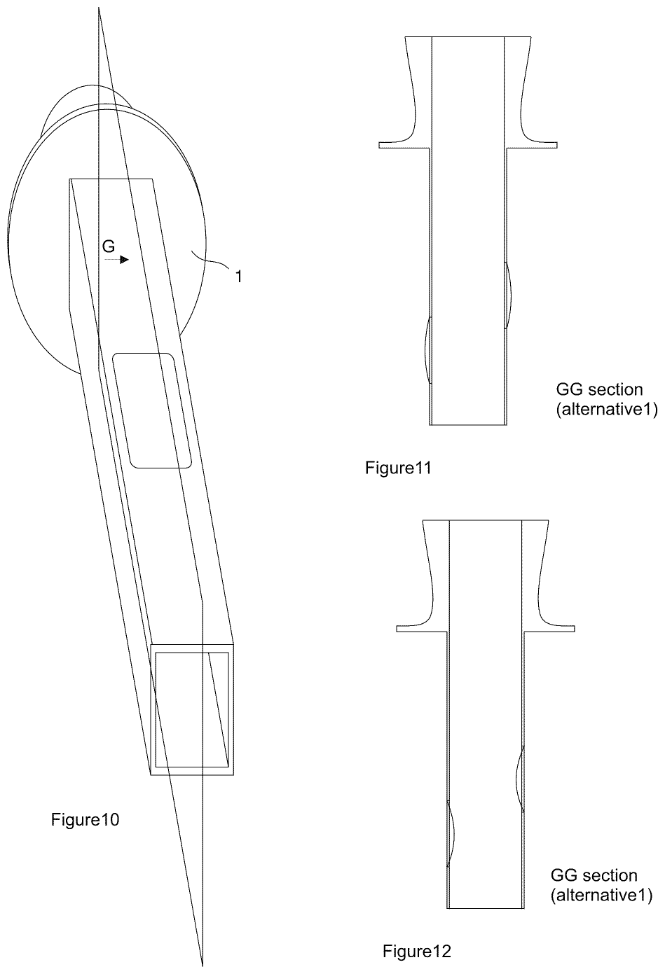

### DEFINITIONS OF COMPONENTS OF THE INVENTION

All elements that provide the wrinkle management developed by this invention are numbered to better explain the subject, and the corresponding of each number is given below.
1. Mouthpiece
2. Airway
3. Filter
4. Support line
5. Support junction point
6. Wrinkle region

### DETAILED DESCRIPTION OF THE INVENTION

The mouthpiece (1) of the invention is produced by pressing the plastic raw material in a female-male mold after it is brought to the melting temperature. Due to the production method and the nature of the raw material, the mouthpiece (1) shrinks, that is, pulls from all directions, depending on the temperature, cooling rate, wall thickness, and dimensions when it is taken from the mold while falling to room temperature. Filters (3) are placed on the apertures on the airway (2) of the mouthpiece (1). While the mouthpiece (1) is produced to adhere to the mouthpiece (1), the filters (3) adhere to the apertures on the airway (2) by contacting the edge points because they are larger in width and length.

Since the filters (3) themselves are cold and the melting mouthpiece raw material is hot during the joining, since the filters (3) do not shrink in the same way during the shrinking of the mouthpiece (1), wrinkle regions (6) are formed on the surface of the filter (3). These wrinkle regions (6) are formed in each production regardless of any order due to their chaotic structure. Therefore, the wrinkle region (6) pattern of the filter (3) on each mouthpiece (1) is different from each other.

The velocity of the air passing through the mouthpiece (1) is measured by means of sound waves moving in the space between the two filters (3) in the airway (2). If the wrinkle regions (6) of the filters (3) get closer to each other, the cross-sectional area in which the air moves are reduced and otherwise it grows. Therefore, even if the velocity of the air is the same, different measurement results can be obtained only due to the differences in the wrinkle regions (6).

By making the wrinkle region (6) predictable and reproducible, similar wrinkles can be obtained as a result of the mass production of the entire mouthpiece. For this purpose, the filter (3) placed in the aperture will be divided by the support lines (4) produced from the same raw material as the plastic raw material used in the production of the mouthpiece (1) to control the wrinkle regions (6). Support lines (4) are formed by filling plastic raw material into gaps in different geometric shapes on the production molds so that they can be formed on the filters (3) during production.

It is expected that the support lines (4) will cool faster than the mouthpiece (1) body, depending on whether they are thick-thin, or flat-high. It is not technically possible for the support lines (4) to cool more slowly than the body of the mouthpiece (1). Support lines (4) showing the same or faster cooling will also ensure that the distribution of the wrinkle regions (6) in the aperture is fixed, as they will adhere to the filter (3). Because the wrinkles in the filter (3) will remain within the wrinkle regions (6) limited by the support lines (4).

As explained above, the main cause of wrinkles is that the width (Xₐₚₑᵣₜᵤᵣₑ) and length (Yₐₚₑᵣₜᵤᵣₑ) of the filter decrease during the cooling time (Δt), and the width (X_{filter}) and length (Y_{filter}) of the filter do not change over time despite the new width (X′ₐₚₑᵣₜᵤᵣₑ) and length (Y′ₐₚₑᵣₜᵤᵣₑ).

$${t}_{1} , {X}_{aperture} , {Y}_{aperture}$$

$${t}_{2} , {X}_{aperture}^{′} , {Y}_{aperture}^{′} ,$$

$${t}_{1} , {X}_{filter} , {Y}_{filter}$$

$${t}_{2} , {X}_{filter}^{′} = {X}_{filter} , {Y}_{filter}^{′} = {Y}_{filter}$$

$$Δ t = {t}_{2 -} {t}_{1}$$

The most important object of the invention is to ensure that the mean (h) of the height values (h₁, h₂, h₃, . . . hₙ) of the said wrinkles in all wrinkle regions (6) in each production remains the same/similar between different mouthpieces.

$$= \frac{{h}_{1} + {h}_{2} + {h}_{3} + … + {h}_{n}}{n}$$

The support lines (4) can cut each other in the aperture. These cut-off points are called the support junction point (5) and are as many as the number of intersecting support lines (4) in the aperture. The support lines (4) themselves and the support junction points (5) prevent the passage of the sound wave that must pass through the filter (3) and measure the air velocity. Therefore, it should be as few as possible by controlling the number and distribution.

The width (X_{support-width}) of the support lines (4), the length (X_{support-length}) of the support lines (4), the distance between the two support lines (4) consisting only of the filter (X_{line-distance}), and the distance (X_{point-distance}) between the support junction points (5) are determined mathematically within the scope of this invention for the types of mouthpieces (1) and filter (3) of different sizes. Thanks to these determinations, even in the use of different filters (3), the shape of their wrinkling will be the same or similar in all wrinkle regions (6) and/or on the filters (3) of the different mouthpieces (1).

The biggest advantage of the similarity of the wrinkle regions (6) is to ensure that the height of the wrinkles (hₙ), that is, the distance value of each wrinkle to the mouthpiece (1) body fluctuates on a certain normal (N) line without showing too much variation.

Although the Xₐₚₑᵣₜᵤᵣₑ-X′ₐₚₑᵣₜᵤᵣₑ and Yₐₚₑᵣₜᵤᵣₑ-Y′ₐₚₑᵣₜᵤᵣₑ values varying over time (t) shorten/reduce the Xₛᵤₚₚₒᵣₜ value at a negligible level, it is inevitable that the filter (3) attached to the support line (4) will be wrinkled as the X_{line-distance} and X_{point-distance} values substantially decrease to X′_{line-distance} and X′_{point-distance} after the passage of time Δt. The support lines (4), which form the grid shape due to the layout, will homogeneously crumple/wrinkle the filter (3) components they take between them while shrinking.

The position of any point on the surface of the filter (3) in three-dimensional space t₁ will change instantly (k, l, m) over time (Δt). If the filter (3) is only crumpled without moving on its plane, only the change of the height component can be taken into account by ignoring the changes of the coordinate data on the plane because they are small. In this case, the same filter (3) point will come to the position (k, l, m′) at the moment of t₂. For each spot, the mean of the “Δmₙ” value or the “hₙ” mean of the heights of the peaks of the wrinkles are close to each other.

$$Δ {m}_{n} ≅ {h}_{n}$$

The largest circle diameter of the point (k,l,m) on any filter (3) that can be drawn without contacting the mouthpiece body (1) or the support line (4) shall be between 1-3 mm.

In the preferred embodiment of the invention, the “X′ₐₚₑᵣₜᵤᵣₑ” value ranges from 6 mm to 10 mm and the “Y′ₐₚₑᵣₜᵤᵣₑ” value ranges from 15 mm to 22 mm. If there are 5 support lines (4) that cut each other in at least 6 points, the total length of the support line (X_{support-total-length}) will be 48 mm-74 mm. The width of the aforementioned support lines (4), that is, the “X_{support-mean-width}” value varies between 0.5 mm and 0.7 mm. The “R_{closed-area}” value, which is the ratio of the filter (3) area in the aperture to the supports, can be calculated as follows.

$${R}_{closed - are} = \% \frac{{X}_{suuport - mean - width} \cdot {X}_{support - total} \cdot 100}{{X}_{aperture}^{′} \cdot {Y}_{aperture}^{′}}$$

Closed area value according to the length and width values of the invention given above:

16.81%<Rₒₚₑₙ₋ₐᵣₑₐ<37.3%

In the preferred embodiment of the invention, when the “X′ₐₚₑᵣₜᵤᵣₑ” value is 9 mm, the “Y′ₐₚₑᵣₜᵤᵣₑ” value is 20.8 mm, X_{support-total-length} is 68.6 mm and the “X_{support-mean-width}” value is 0.6 mm, it will be:

R_{closed-area}=%21.9

This value will enable mouthpiece (1) to be taken under control without creating a problem of measuring the wrinkle of the filter (3) after its natural shrinkage.

In the selection of filter (3), a filter (3) with an open area between 10% and 50% can be used regardless of fiber thickness. In the preferred embodiment of the invention, a filter (3) with an open area between 20-25% will be used.

In the preferred embodiment of the invention, the support lines (4) can be placed in the aperture in different angles and directions or parallel to each other without forming at least 3 horizontal and 2 vertical components and at least 6 support junction points (5). The positions of these support junction points (5) will be close to the center of the aperture.

1. A mouthpiece for allowing the filters for protecting against contamination during passage of air flow by sensors in pulmonary function tests to homogeneously distribute wrinkles of mouthpieces during production, the mouthpiece comprising:
a body formed from plastic raw material that is pressed in a female-male mold after it is brought to a melting temperature,
two filters for allowing measurement of a speed of air passing through an airway in the mouthpiece based on a speed of sound waves moving in a space between the two filters,
plural wrinkle regions formed on a surface of the two filters, wherein shrinkage of the two filters is different from shrinkage in the molded plastic raw material as a width (Xₐₚₑᵣₜᵤᵣₑ) and a length (Yₐₚₑᵣₜᵤᵣₑ) at a moment t₁ reach new width (X′ₐₚₑᵣₜᵤᵣₑ) and new length (Y′ₐₚₑᵣₜᵤᵣₑ) values at a moment t₂ after a cooling time of Δt;
plural support lines that lower a mean of height values (h₁, h₂, h₃, . . . hₙ) in the plural wrinkle regions to make wrinkles in each wrinkle regions predictable and reproducible, the support lines being produced from the plastic raw material used in the body, wherein the support lines are arranged to divide the two filters.

2. A mouthpiece according to claim 1, wherein at least one of the two filters has a closed area in the range of 16.81%<R_{closed-area}<37.3% according to the formula
$${R}_{closed - area} = \% \frac{{X}_{suuport - mean - width} \cdot {X}_{support - total - length} \cdot 100}{{X}_{aperture}^{′} \cdot {Y}_{aperture}^{′}}$$
when the “X′ₐₚₑᵣₜᵤᵣₑ” value is between 6 mm and 10 mm, the “Y′ₐₚₑᵣₜᵤᵣₑ” value is between 15 mm-22 mm, the total length (X_{support-total-length}) reached by at least one of the plural support lines is between 48 mm-74 mm and the width (X_{support-mean-width}) of the plural support lines is between 0.5 mm-0.7 mm.

3. A mouthpiece according to claim 1, wherein the plural support lines include at least five support lines that cut the body into at least six support junction points with at least three horizontal components and at least two vertical components.

4. A mouthpiece according to claim 3, wherein at least one of the two filters has a closed area with a value of R_{closed-area}=%21.9 according to the formula
$${R}_{closed - area} = \% \frac{{X}_{suuport - mean - width} \cdot {X}_{support - total - length} \cdot 100}{{X}_{aperture}^{′} \cdot {Y}_{aperture}^{′}}$$
when the “X′ₐₚₑᵣₜᵤᵣₑ” value is 9 mm, the “Y′ₐₚₑᵣₜᵤᵣₑ” value is 20.8 mm, X_{support-length} is 68.6 mm and “X_{support-width}” is 0.6 mm.

5. A mouthpiece according to claim 1, wherein the plural support lines include intersecting support lines, which are placed to limit a diameter of a largest circle that is drawn without contacting the body or one of the plural support lines between 1-3 mm.

6. A mouthpiece according to claim 1, wherein the plural support lines include parallel support lines, which are arranged to limit a diameter of a largest circle that is drawn without contacting the body or one of the support lines between 1-3 mm.

7. A mouthpiece according to claim 1, wherein the plural support lines are distributed in an aperture at different angles and directions to limit a diameter of a largest circle that can be drawn without contacting the body or one of the plural support lines between 1-3 mm.
